(19) United States
(12) Patent Application Publication
(10) Pub. No.: US 2009/0043330 A1
(43) Pub. Date: Feb. 12, 2009

(54) Embolic protection devices and methods

(75) Inventor: John Thao To, Newark, CA (US)

Correspondence Address: Katherine N. Addison Esq., 501 Marie Avenue, Los Angeles, CA, 90042-1305, US

(73) Assignee: SPECIALIZED VASCULAR TECHNOLOGIES, INC., Newark, CA (US)

(21) Appl. No.: 12/189,538

(22) Filed: Aug. 11, 2008

Related U.S. Application Data
(60) Provisional application No. 60/964,045, filed on Aug. 9, 2007.
(60) Provisional application No. 60/993,329, filed on Sep. 11, 2007.
(60) Provisional application No. 61/001,217, filed on Oct. 31, 2007.

(51) Int. Cl. A61M 29/02; A61M 29/00

(52) U.S. Cl. 606/194; 606/198; 128/898; 606/200

(57) ABSTRACT

The present invention provides a PreStent comprising a sheath and frame designed for preparing a vessel passageway for the subsequent delivery of a stent. The PreStent can be self-expanding or balloon-expandable. Also provided is a supporting system for delivering the PreStent safely. The supporting system includes a delivery catheter, one or more occlusion balloon, optionally one or more dilation balloon, and a retention sheath for the self-expanding type of PreStent. The PreStent of the present invention is flexible for use with a variety of stent and guidewire models such that it can easily be incorporated with existing devices to improve stenting procedures.

### BACKGROUND OF THE INVENTION

1. Field of the Invention

The present invention is directed at devices and methods for preventing an embolism when performing intravascular procedures such as percutaneous transluminal coronary angioplasty (PTCA) or percutaneous transluminal angioplasty (PTA). More specifically, the invention provides a device to precede the deployment of a stent that compresses and stabilizes plaque against a vessel wall and protects the inner lumen of a vessel from abrasion by the struts of the stent during placement. The method is directed at insuring the safe placement and maintenance of an intravascular prosthesis.

2. Description of the Related Art

Presently, embolic protection devices are used for procedures that entail a high risk of embolization with adverse consequences. These procedures include carotid artery stenting (CAS), renal artery stenting (RAS), and vein graft stenting (VGS). Adverse consequences can include seizure, stroke, and even death incited when plaque dislodged from a lesion being treated and traversed enters the bloodstream and travels downstream to disrupt blood flow and oxygen from reaching critical organs. Conventional approaches include distal balloon occlusion (Percusurg GuardWire) with aspiration, filter devices (Angioguard, Filterwire, EmboShield, Spider), and flow reversal devices (ArteriA).

The distal balloon occlusion device and the filter device suffer from having to place a bulky device far distal to the target lesion which can cause complications such as spasm.

The far distal positioning of these devices permits embolization out a branch just distal to the lesion and proximal to the embolic protection device. This is especially a problem with RAS. These devices are also bulky and stiff which increases their risk of inducing embolization upon crossing the lesion in the first place if they inadvertently tear a side of the vessel wall. The filter devices have pore sizes that must be big enough (100 microns) to not get clogged up. As a result, the pores allow many smaller particles, including freed plaque particulates, to pass through. Although smaller particulates of plaque may not be capable of inducing embolization when dispersed in the bloodstream, they may collectively cause problems when aggregated upon distal lesions or upon embolic protection devices and prostheses.

Other weaknesses of the filter devices are their susceptibility to enabling backflow and differential blood flow rates. Backflow can result from clogging of the filter or from compression of the filter to move or remove it. If the filter does not have an adequate sealing mechanism for the debris-catching compartment then there is the risk of trapped debris being squeezed through the opening when the filter is compressed. Filter devices also suffer from retrieval failures at the end of a procedure.

Traditional balloon occlusion devices also encounter problems at the end of a procedure including aspiration inadequacy as they are inflated and deflated. Aspiration inadequacy can delay drainage and cause embolization. Placement and inflation of traditional (uniformly shaped, non-toroidal) distal balloon occlusion devices interrupts flow.

The flow reversal device is bulky (10 French Outer Diameter (OD)) and also interrupts flow.

Using all of these devices requires many extra steps that complicates the procedure and extends procedure time significantly. All of these devices only protect from embolization acutely. The stents with which these devices are used have open spaces between the struts that allow for particles to loosen from the lesion and embolize post-procedure. Typically, this occurs around 3 days post-procedure in CAS and presents a significant subacute embolization problem that is currently not addressed.

More recent alternative devices and methods have attempted another approach to preventing embolization that involves trapping plaque at the site of the lesion with a tubular membrane outside of the stent.

United States Patent No. (hereinafter USP) U.S. Pat. No. 6,592,616 by Richard S. Stack, et al. and assigned to Advanced Cardiovascular Systems, Inc. discloses a tubular net of blood permeable and biocompatible material with expandable members attached to each end. Since the net does not form a continuous surface, plaque particulates could pass through it. Further the net does not have its own frame structure other than the expandable members at the ends and therefore it depends upon a stent or balloon to maintain an expanded configuration. Consequently, the central portion of the net between the expandable members at the ends may be susceptible to sagging and difficult to conform precisely to the vessel's inner luminal wall. This may result in the formation of pockets that alter hemodynamics and encourage embolization.

U.S. Pat. No. 6,699,276 by David Sogard, et al. and assigned to SciMed Life Systems, Inc. discloses a composite medical device comprising a support structure (i.e. a radially expandable stent), a porous non-textile polymeric membrane, and a thermoplastic anchoring means for attaching the membrane to the structure. As with the device of USP '616, the membrane itself does not have a frame, thereby making it dependent upon the stent. The emphasis of this patent on the anchoring means reinforces this dependency. This could be problematic if the insertion of the membrane is to precede implantation of the stent, with the same foreseeable problems as for the frame-less net of USP '616: sagging, poor adhesion to vessel's inner lumen, air pockets, etc.. The present invention improves upon these designs by providing a thin stand-alone, self-supporting PreStent with its own coiled framework to avoid these problems.

### BRIEF SUMMARY OF THE INVENTION

The present invention is designed to facilitate the safe insertion of an intravascular prosthesis used to maintain vascular patency. The system of the present invention includes a PreStent component of an atraumatic flexible sheath that abuts a vessel wall and a supporting frame structure of coils. The system also includes other components such as insertion tools (i.e. delivery catheter, retention sheath for self-expanding embodiment of PreStent), dilation components (i.e. balloons, toroidal balloons), and occlusion components (i.e. dumbbell-shaped balloons). The invention is designed to improve both the safety and efficacy of stenting by reducing the risk of embolism during implantation, acutely post-implantation and in the long-term while controlling immune responses encouraging endothelization in the vicinity of the lesion and stent.

The present invention overcomes the problems of the reference art including: far distal placement, bulkiness, rigidity, retrieval failures, aspiration inadequacy, filter clogging, etc. The PreStent of the present invention covers the entire length of the lesion and because it is self-supporting can extend beyond the proximal and distal margins of the stent. The plaque-trapping device disclosed in U.S. Pat. No. 6,592,616 (discussed above) is notably shorter than the stent (see claim 5). The ability of the PreStent position to both encompass and extend beyond the position of the lesion avoids the problem of embolization in side branches immediately distal (or proximal) to the lesion margins as associated with bulky embolic protection devices placed far distal to the lesion. Since the stent is the primary source of radial support for the vessel and separate dilation balloons are used for vessel expansion, the PreStent can be made to have lower profile and can be more elastic allowing it to collapse to a smaller profile for delivery but still deploy to a large diameter. The absence of filter, flow reversal, or other more complicated structural elements eliminates bulk. The flexible atraumatic membrane and springy coiled frame structure are independently circumferentially slidable to provide an adaptable ergonomic fit rather than trauma-prone rigidity. Since the PreStent remains after placement and is designed to be naturally incorporated into the vasculature (i.e. via bioabsorption, biodegradation, bioerosion, etc.) with time as endothelization progresses, retrieval failure is not a possibility. Aspiration inadequacy is also not an issue since the re-opening of the vessel maintains hemodynamics and sufficient channel volume to permit the insertion of separate aspiration devices as needed. The aspiration means can also be provided around the outer circumference of separate toroidal balloons distinct from but complementary to the PreStent component.

The present invention addresses all of the issues associated with conventional embolic protection devices of the reference art by providing a thin stent-like structure covered with a thin porous sheath that can be deployed to covered the lesion as a first step in the stenting procedure and left as an implant to protect from sub-acute embolization. A similar concept has been conceived by Gifford et al. (see U.S. Pat. No. 6,383,171) but only involved a sheath with anchors on the ends. Unlike the present invention, Gifford's sheath can collapse inward and get caught up by a stent that is delivered inside subsequently. The self-supporting springy coiled frame structure and affiliated protective membrane of the PreStent according to the present invention avoids these problems.

Unlike the present invention, previous designs of covered stents would not work for this application because they are not designed to be flexible enough, to be collapsed to a small enough profile, to seal well against the vessel wall, nor to easily expand to a diameter much larger than the collapsed diameter.

Other aspects of the present invention include non-implant devices and approaches that may complement or supplement the use of the PreStent. These approaches address some of the acute problems described above.

One device is an introducer or angiography catheter with a proximal flow occlusion mechanism (i.e. balloon, basket, etc.) that allows for the introduction of embolic protection devices (EPDs) to pass through a dilatable tapered tip. Another is a temporary tubular mesh or toroidal balloon to cover the lesion to allow safe pre-dilation and passage of other EPDs safely and easily. Finally, a temporary cover mesh or toroidal balloon can be combined with a dilation balloon and stent for an efficient procedure.

### BRIEF DESCRIPTION OF THE SEVERAL VIEWS OF THE DRAWINGS

FIG. 1A shows a close-up side view of the PreStent of the present invention according to one embodiment with (i) a frame having diagonally criss-crossing struts and flared ends for apposition against a vessel wall proximal and distal to a lesion and (ii) a continuous porous sheath that covers the spaces between the struts.

FIG. 1B shows a close-up side view of the PreStent of the present invention according to another embodiment with (i) a frame having intermittent groups of tight-pitch coiled struts connected by sinusoidal links and (ii) a continuous porous sheath that covers the spaces between the struts.

FIG. 1C shows a close-up side view of the PreStent of the present invention according to another embodiment with (i) a frame having a continuous series of tight-pitch coiled struts connected by sinusoidal links and (ii) a continuous porous sheath that covers the spaces between the struts.

FIG. 1D shows a side view of the PreStent of the present invention according to another embodiment with (i) a frame having a variable series of tight-pitch coiled struts interspersed with non-tight-pitch sinusoidal struts (in which the sinusoids are oriented perpendicular to the longitudinal axis of the stent) connected by sinusoidal links (in which the sinusoids are oriented parallel to the longitudinal axis of the stent) and (ii) a continuous porous sheath that covers the spaces between the struts.

FIG. 2B shows a cross-sectional side view of the self-expanded PreStent and Delivery System of FIG. 2A, illustrating the delivery catheter's hollow lumen for accommodation of a guide wire.

FIG. 3A shows an in situ side view of the PreStent extended across a plaque-filled lesion within a vessel along with the PreStent Delivery System including a tapered tip catheter, retention sheath and proximal occlusion balloons.

FIG. 3B shows an in situ side view of the PreStent and Delivery System of FIG. 3A with the onset of PreStent deployment at the distal end as the retention sheath is directed proximally.

FIG. 4A shows an in situ side view of the PreStent, fully deployed across the length of the lesion upon an expansion balloon over the delivery catheter and guide wire with the proximal occlusion balloons still in place.

FIG. 4B shows an in situ side view of the PreStent, fully deployed across the length of the lesion after the PreStent Delivery System (catheter, guide wire, expansion balloon, and occlusion balloons) has been removed.

FIG. 4C shows an in situ side view of a deployed stent within the deployed PreStent both spanning the length of the lesion within a vessel.

FIG. 5A shows an in situ side view of an alternative PreStent that is not self-expanded but requires a separate expansion balloon (compared to the self-expanded PreStent shown in FIG. 2 and FIG. 3) but does not require a retention sheath because premature auto-expansion is not a risk.

FIG. 5B shows an in situ side view of the non-self-expanded PreStent of FIG. 5A, fully deployed across the length of the lesion upon a separate expansion balloon over a delivery catheter and guide wire and with proximal occlusion balloons still in place.

FIG. 5C shows an in situ side view of the non-self-expanded PreStent of FIG. 5A, fully deployed across the length of the lesion after the PreStent Delivery System (catheter, guide wire, separate expansion balloon, and occlusion balloons) has been removed.

FIG. 6 shows a direct stenting approach in which a stent is loaded on the waist of a dumbbell-shaped balloon with distal and proximal ends that expand to occlude blood flow and a toroidal configuration to permit blood flow through its center.

FIG. 7 shows how aspiration and flushing functions can be provided through the outer walls of the porous occluder dumbbell-shaped balloon as it traverses a plaque-filled lesion to remove emboli while the inner lumen maintains the natural hemodynamic environment to permit blood flow.

FIG. 8A shows one alternative for deploying a PreStent at a bifurcation, in which a first PreStent is deployed in a distal branch requiring treatment followed by the deployment of a second PreStent in a common branch, leaving open at least one peripheral distal branch that does not require treatment.

FIG. 8B shows another alternative for deploying a PreStent at a bifurcation, in which a single PreStent is deployed across the common branch and one peripheral branch.

FIG. 9 shows one possible strut pattern for the PreStent frame, in its collapsed and expanded position, with the variable geometry and curves providing radial support, distributed tension, and a smooth collapse and expansion process.

### DETAILED DESCRIPTION OF THE INVENTION

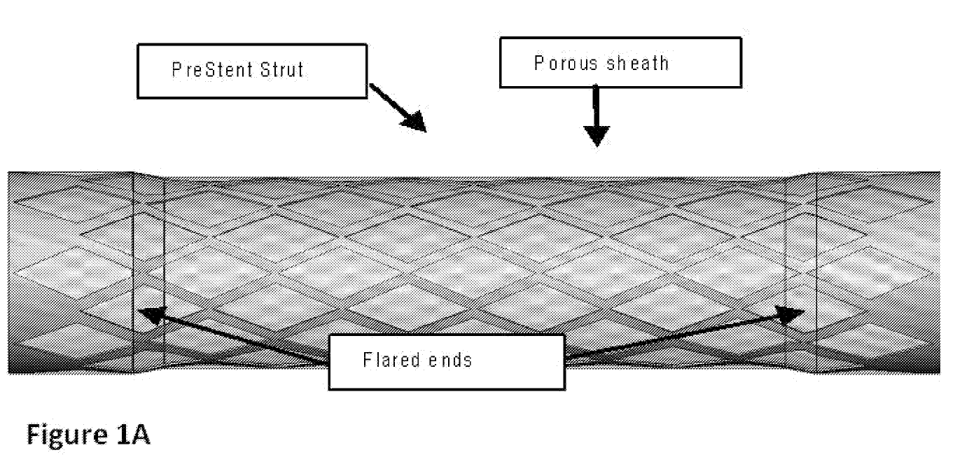

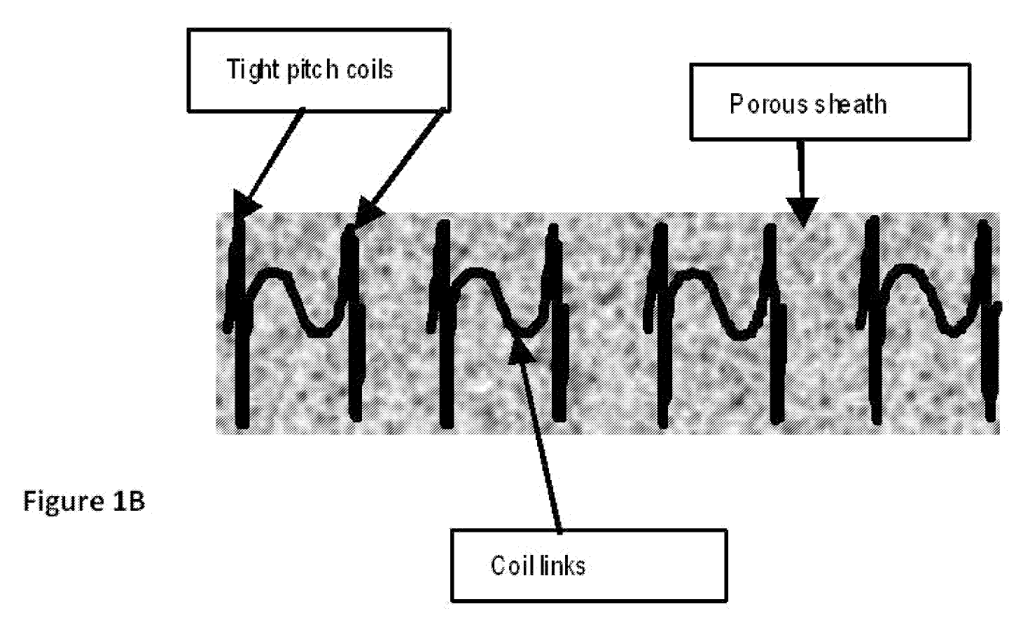

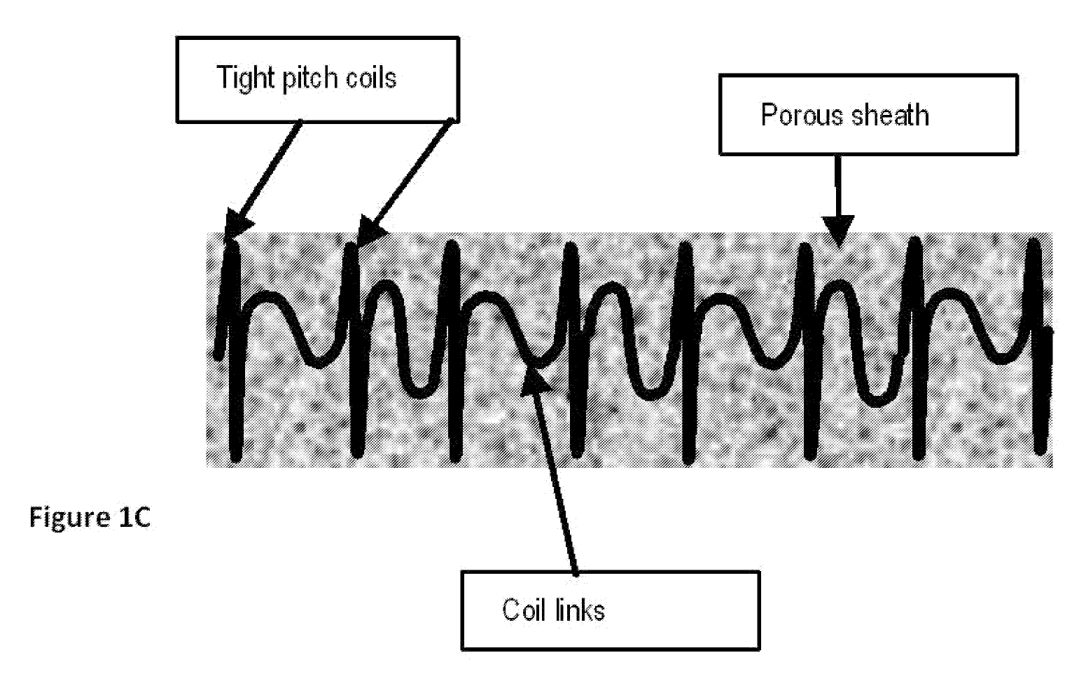

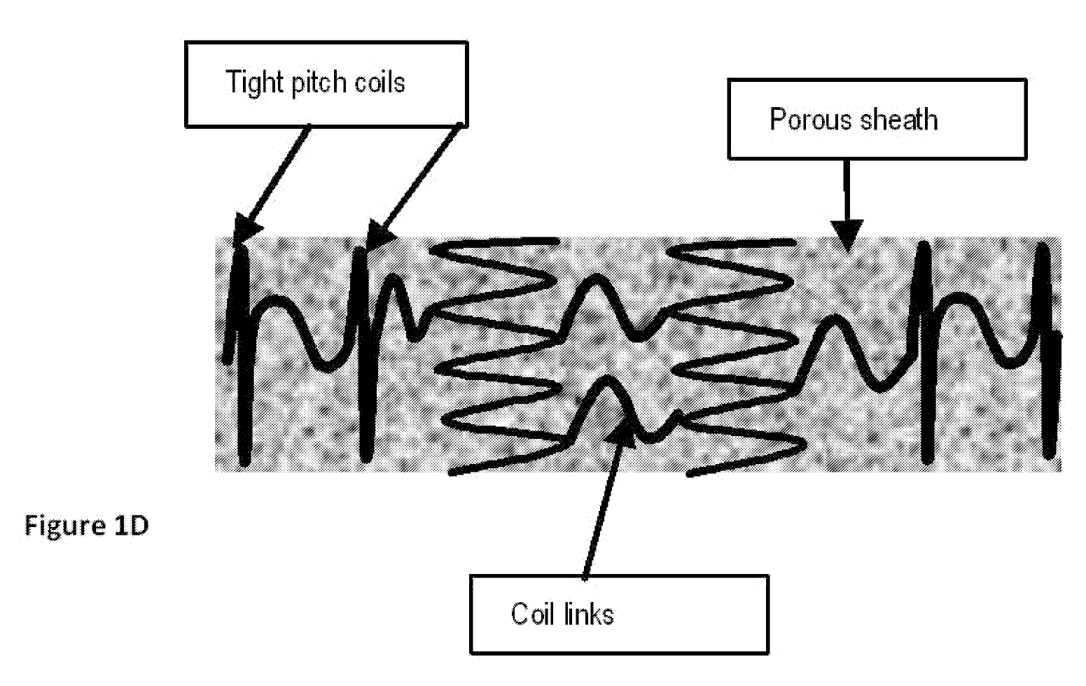

The first aspect of the present invention for embolic protection is a structure comprising a thin, expandable stent-like frame covered with a porous, expandable sheath (see FIG. 1A). The preferred frame design is shown in FIG. 1B. This structure acts as a precursor to a stent and is called a PreStent. Its main function is to cover the lesion completely so that no embolization can occur from subsequent operations such as balloon dilatation and/or stenting. Since it is left as an implant post-stenting, it also prevents embolization from occurring post-procedure. Post-procedure embolization is a possibility because most stents have abrasive struts that can dig into a vessel wall to dislodge plaque coupled with large openings between the struts that allow dislodged plaque particles to enter the bloodstream.

The step of crossing the lesion and deploying an EPD is replaced with the deployment of a PreStent with a low profile delivery device. All of the troublesome issues associated with the procedures involving conventional EPDs are eliminated. In particular, the interference of conventional EPDs with normal hemodynamics is avoided as the PreStent system of the present invention allows for good blood perfusion through the vessel during the procedure. Since there is nothing to retrieve, all the complications associated with EPD retrieval post-stenting are also eliminated. Aspiration difficulties such as fluid flow obstruction are avoided by a self-supporting, expandable frame element and the incorporation of aspiration channels on separate components such as on the outer circumference of toroidal balloons. Sub-acute embolization is eliminated because of the retention of the porous sheath covering the lesion and protecting the inner vessel post-procedure.

The PreStent can be designed to be very thin and flexible because it does not have to act as a scaffold to prop the vessel open like a regular stent. There are multiple benefits to having a stent-like structure that has the wall and strut thicknesses at a fraction of those needed for a stent. First, thinner PreStent can be collapsed to a smaller diameter (<1 mm) for delivery through tight lesions without getting stuck or causing embolization. When a stent is collapsed, there is beam bending on the strut. The more the stent is collapsed relative to the final deployed diameter, the greater the bending on the strut. According to beam bending theory, the thinner a beam, the less strain under a given bend of the beam. Less strain is better for structural integrity. For example, in a preferred embodiment in which the stent is self-expanding, the struts are made out of a shape memory material such as Nitinol (NiTi). Past 8% strain, Nitinol tends to lose its ability to fully recover. In the case of the PreStent, where the strut thickness is small, the strain is also small and the structure can be collapsed to a small diameter without exceeding 8% strain so that when the PreStent is free to expand, it can fully expand to its larger diameter range. In contrast, it would be easier to cause the thicker struts of a traditional stent to exceed the strain tolerance for recoverability. This results in a compromise between collapseability and expansion with thicker struts.

The thinner strut and wall thickness of the PreStent also makes the overall structure more flexible and conformal to the lesion and vessel. In delivery, the catheter tip loaded with the PreStent is also flexible and is important for crossing the lesion.

The preferred frame design is a series of pairs of tight-pitch helical coils connected by sinusoids in a continuous fashion (see FIG. 1B). Unlike a conventional stent frame, where uniform scaffolding with uniform radial strength throughout the whole body is needed, the PreStent frame does not need to be uniform in radial strength. Rather, the PreStent frame needs to be following: 1) maximally flexible in bending for ease of delivery; 2) the ends need to approximate a continuous hoop after it expands to seal well against the vessel for blocking embolization; 3) have some radial strength along the whole length to keep the sheath from collapsing inward before stenting; and 4) the frame element(s) must be free to move relative to each other during expansion without being overly constrained by adhesion to the sheath so as to cause the sheath to wrinkle, delaminate, or tear. The design of the frame as shown in FIG. 1B is optimized for these four criteria. Alternative embodiments include a frame with a series of coils all connected by sinusoids or zigzags, and a frame with coils on the ends and sinusoids or zigzag rings in between along the length of the PreStent (see FIG. 1B and FIG. 1C). Finally, the frame body can incorporate the design of any existing with tight pitched coils on the ends.

The PreStent can be self-expanding or balloon expandable. For the self-expanding embodiment, the struts are preferably made of shape memory or superelastic metals such as Nitinol or shape memory polymers including aliphatic polyesters especially poly(etherester)s, as well as L,L-dilactide, diglycolid, and p-dioxanoile. Preferable materials for the stent struts in a balloon expandable embodiment include stainless steel (316L), titanium alloys (Ti6Al4V), tantalum, or cobalt chromium. The struts can also be constructed of biodegradable or bioresorbable materials including magnesium, polylactic acid (PLA) compounds, polyglycolic (PGA) compounds, polyhydroxybutyriate (PHB), polydioxanone, polyanhydrides, poly-ortho esters, polyiminocarbonates, any blend of these polymers, or any co-polymers of these polymers (i.e. PGA-PLA). Stiff polymers like polyetheretherketone (PEEK), polyimide, polycarbone, fluoropolymers (i.e. Teflon, ETFE (Ethylene tetrafluoroethylene)), liquid crystal polymers (p-hydroxybenzoic acid), and parylene can also be used. The frame may also be made of organic materials such as bone or tendons. Other frame materials may include ceramics (zirconia, boron carbide, boron nitride, silicon carbide, silicon nitride), carbon fiber, and glass. The frame can also be constructed of a combination of multiple types of the aforementioned materials.

Radioopaque markers can be coated on all the struts or just the ends. They can also be welded or attached to the strut. The markers can be made of gold, tantalum, iridium, tungsten, or platinum. Radioopaque compounds such as bismuth, barium sulfate or the aforementioned compounds can also be blended in with the frame material.

The frame body, coils, or just the struts can be coated or impregnated with drugs or molecules to minimize thrombus formation including any anticoagulants or thrombolytics such as phosphorylcholine (PC), heparin, dextran, chlopidegrel, ticlopidine, GPVI antagonists, antagonists to the platelet adhesion receptor (GP1b-V-IX), antagonists to the platelet aggregation receptor (GPIIb-IIIa), enoxaparin, dalteparin, hirudin, bivalirudin, argatroban, danparoid, and/or TFPI (Tissue Factor Pathway Inhibitor). Any part of the frame can also be coated or impregnated with pro-endothelization substances such as vascular endothelial growth factor (VEGF), angiopoietin-1, and/or phosphorylcholine. Any combination of the above mentioned therapeutic substances can be used.

The strut thickness is preferably 0.0005″-0.003″ (0.0127-0.0762 mm). The strut width is preferably 0.001″-0.007″ (0.0254-0.1778 mm). More preferably, the strut width is 0.002″-0.005″ (0.0508-0.127). The struts can be round or wireform and the diameter is preferably 0.001-0.003″ (0.0254-0.0762 mm). The strut structure can be generated by cutting thin wall tubes with EDM (electrical discharge machining) or laser. Wireforms or thin rings can be welded together. Round or ribbon wireforms can be woven in a mesh or braid. A flat sheet can also have patterns cut into it using chemical etching, photoetching, EDM, and/or laser and then be folded into a tube and shape set with heat or welded at the open ends.

The sheath can be laminated around the entire frame body or just the struts to encapsulate. Alternatively, the sheath can be molded as a cover on only the outside of the frame body or just the struts. The sheath can be porous with a preferred pore size of 10-50 microns. Pores are important to allow tissue to grow through and heal over the implant on the inside. Encapsulation of the PreStent and the stent with neointima is important for preventing adverse immunological responses (i.e. thrombosis) in the long term. (See also co-pending commonly owned U.S. patent application Ser. No. 12/128,533 directed to “Coatings for promoting endothelization of medical devices”.) The thickness of the sheath is preferably 10 microns to 0.003″ (0.0762 mm). The sheath is preferably made of bioresorbable or biodegradable polymers such as polylactic acid (PLA), poly L-lactic acid (PLLA), poly glycolic acid (PGA), polycaprolactone, polyetheresters, silk or modified collagen. It can also be made of non bioresorbable material but nonreactive expandable materials such as ePTFE (polytetrafluoroethylene). Bismuth or Barium Sulfate can be compounded with these polymers to give it radioopacity. The sheath can also be made of an elastomer (such as silicone, polyurethane, or isoprene) to allow it to expand over a wide range. Since silicone is hydrophobic, it can be degraded by fatty acids and triglycerides which are present in blood. At such a thin thickness post expansion, it can easily degrade and allow tissue to grow through without the need to have pores. The elastomers can be applied to encapsulate the strut structure by controlled dispensing of liquid form from a pressurized needle tip onto a stent rotating on a fixture. If pores are desired, plasma etching can be performed to introduce microscopic pores. Even drug coating can be added by vapor deposition process such as parylene coating with heparin. The sheath material and/or drug can be formed by spraying on using pressurized nozzle or ultrasound spray coating technology.

The sheath also provides an easy means for local drug delivery. It can be coated or embedded with anticoagulants including heparin, dextran, hirudin, phosphorylcholine (PC), and/or chlopidegrel to prevent thrombosis. It can also be coated or embedded with immunological suppressants or antiproliferatives, including taxol, everolimus and rapamycin, to minimize restenosis.

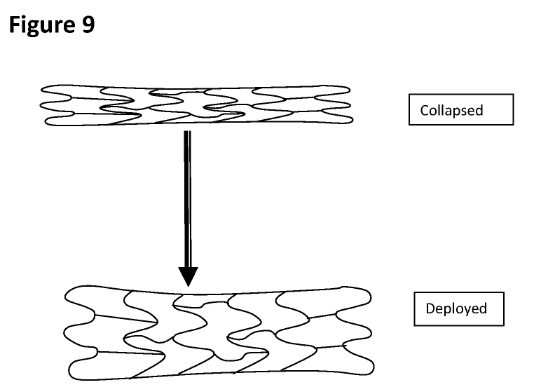

The strut structure is designed so that the ends can flare out to appose against the vessel proximal and distal to the lesion to seal up the path for embolization (FIG. 1). The PreStent is preferably designed to expand from 2-9 mm in diameter. The strut pattern shown in FIG. 1 is only for the purpose of general illustration. There are many preferred patterns that can be used and are shown in FIG. 9.

One way to create the frame is to bend a wire or ribbon into the shape of the frame and setting it with heat or cold work. It can also be generated by cutting thin wall tubes with EDM or laser. Wireforms or thin rings can be welded together. Round or ribbon wireforms can be woven in a mesh or braid. A flat sheet can also have cut patterns using chemical etching, photoetching, EDM, and/or laser and folded into a tube and shape set with heat or welded at the open ends. Polymers can be dispensed from a syringe or nozzle in liquid form in a controlled pattern of the frame over a round mandrel. A mandrel can be masked with a negative pattern of the stent frame and vapor deposition can be used to apply the material to the madrel to form the frame. Stereolithography or fuse deposition can also be used to lay down the frame material. The frame can also be molded. Ultrasonic spray can also be used to form the frame.

The sheath can cover the frame on the outside or be an inner layer and outer layer laminating the frame in between. The sheath is not attached to the coils to allow the coils to freely unwind. In the case where the frame is laminated in the sheath, the inner and outer layers of the sheath are only attached to each other between the coils and beyond the ends of the coils. The layers can be fused together with energy, or it can be glued together or attached to each other with threads (stitch). In the case of the sheath cover on the outside the sheath can be folded over the end coils as a cuff and stitched, glued, or fused on the end.

The sheath pore size is preferably 10-80 microns. Pores are important to allow tissue to grow through and heal over the implant on the inside. Encapsulation of the PreStent and Stent with neointima is important for preventing adverse immunological response long term such as thrombosis. The thickness of the sheath is 5 micron to 0.003″ (0.0762 mm).

The sheath is preferably made of bioresorbable or biodegradable materials including magnesium alloys, hydroxyapatite, and polymers such as polylactic acid (PLA) compounds, polyglycolic (PGA) compounds, polycaprolactone, polyhydroxybutyriate (PHB), polydioxanone, polyanhydrides, poly-ortho esters, polyiminocarbonates, polyetheresters, any blend of these polymers, or any co-polymers of these polymers (i.e. PGA-PLA). The sheath can also be made of natural or synthetic silk or modified collagen. Alternatively, the sheath can be made of non-bioresorbable materials including nonreactive, expandable fluoropolymers (i.e. ePTFE, ETFE).

The sheath can also be made partially or entirely of elastomers to allow it to expand over a wide range. Exemplary elastomers include silicone, polyurethane, polyzene-F, and/or isoprene. Elastomers are generally hydrophobic, permitting an elastomeric sheath to gradually be degraded by fatty acids and triglycerides present in a blood vessel. Since the sheath has such a thin thickness post expansion, it easily degrades to allow tissue to grow through even without pores. The elastomers can be applied to encapsulate the strut structure by controlled dispensing of liquid from a pressurized needle tip onto a frame rotating on a fixture.

Finally, the sheath can be made of thin fibers of Nitinol, stainless steel, titanium alloys, tantalum, tungsten alloys, carbon fibers, or glass fibers.

Like the struts and frame body, the sheath can also be made radioopaque, for example, by compounding bismuth or barium sulfate with the primary material(s).

Pores in the polymers of the sheath can be created with a variety of well known processes including: air mixing in the extrusion or molding of the material, laser, chemical etching, and/or plasma etching. Thin fibers, 2 microns to 0.002″ (0.0508 mm) thick, of the sheath material can be woven, braided, fused, adhesively bonded and/or knitted to form a porous mesh that is flexible. Vapor deposition of binding material (i.e. parylene) may be used to hold the fibers together.

In a preferred embodiment, the fibers are laid down in aligned fashion to encourage endothelization. For example, an inner layer of the sheath may have fibers aligned longitudinally or approximately parallel to the length of the vessel to encourage endothelial cells to develop on the inner surface.

The sheath also provides an easy means for local drug delivery. It can be coated or impregnated with drugs or substances to minimize thrombus formation including any anticoagulant, heparin, chlopidogrel, ticlopidine, GPVI antagonists, antagonists to the platelet adhesion receptor (GP1b-V-IX), antagonists to the platelet aggregation receptor (GPIIb-IIIa), enoxaparin, dalteparin, hirudin, bivalirudin, argatroban, danparoid, and/or TFPI. The sheath may also be coated or impregnated with pro-endothelization substances including vascular endothelial growth factor (VEGF), angiopoietin-1, and/or phosphorylcholine. Any combination of therapeutic agents, including those mentioned above, can be used to minimize thrombus formation.

The sheath may also be coated or embedded with immunological suppressants or anti-proliferative drugs including taxol, everolimus and/or rapamycin to minimize restenosis.

Other substances for this purpose include Biolimus, Zotarolimus, Tacrolimus, basic fibroblast growth factor (bFGF), rapamycin analogs, antisense dexamethasone, angiopeptin, Batimistat™, Translast™, Halofuginon™, acetylsalicylic acid, hirudin, steroids, ibuprofen, antimicrobials, antibiotics (i.e. Actinomycin D), tissue plasma activators, and/or agents that affect VSMC (vascular smooth muscle cell) proliferation or migration (i.e. transcription factor E2F1). It can also be coated with polyzene-F or PTFE.

These anti-thrombus and anti-proliferative drugs can be incorporated into the PreStent material by: (i) blending the bioactive agent(s) in with the resin during extrusion or molding, (ii) soaking the sheath in a solution of drug and vaporizing the solvent, (iii) injecting or dispensing the drug solution onto the sheath with a nozzle or a syringe, (iv) dissolving the drug in one or more volatile solvent and spraying on, and/or (v) vapor deposition.

In an alternative embodiment, thicker fibers of the frame material (0.0005″-0.002″/0.0127-0.0508 mm) can be woven, braided, fused, adhesively bonded and/or knitted to form a thin porous mesh with micropores (10-100 microns) that is flexible and has radial strength once expanded. This would eliminate the need for having a separate frame as well as a sheath. In essence, the two are combined here.

The sheath and/or frame is preferably negatively charged or hydrophobic on the outer surface to minimize development of thrombus.

The PreStent is preferably designed to expand with a 3-4 fold increase in diameter (i.e. from 2.5-9 mm).

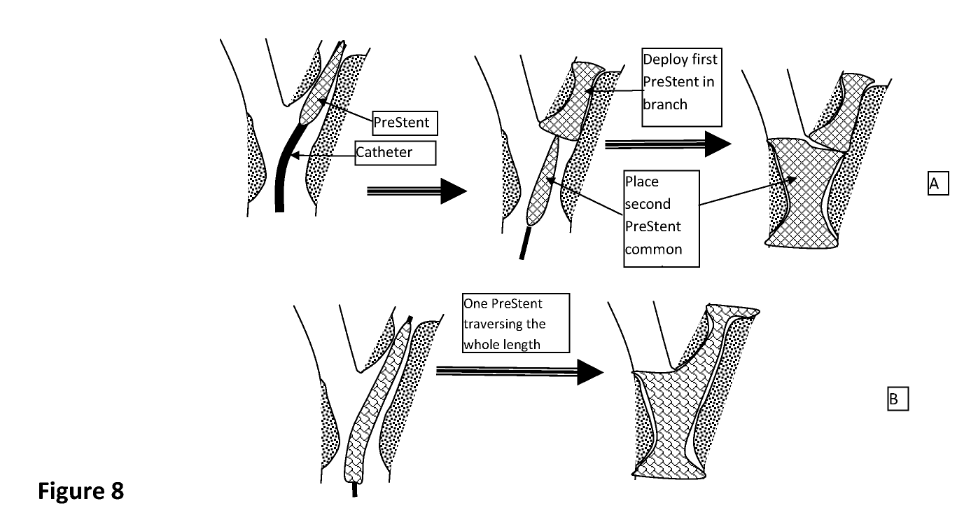

Often the lesion is at a bifurcation, as is frequently seen in CAS where the common carotid artery (CCA) split into the internal carotid artery (ICA) and the external carotid artery (ECA). Clinically it is important to treat the ICA. The ECA isn't as important. One option is to deploy the PreStent across the bifurcation and block off the ECA (see FIG. 8B). The sheath can be biodegradable or bioerodable and disappears within 1 week and allows flow back into the ECA. Another option is to deploy a PreStent in the ICA and another one in the CCA (see FIG. 8A). The risk of embolization for either method is low if the PreStent is self-expanding because the distal vessel is covered first.

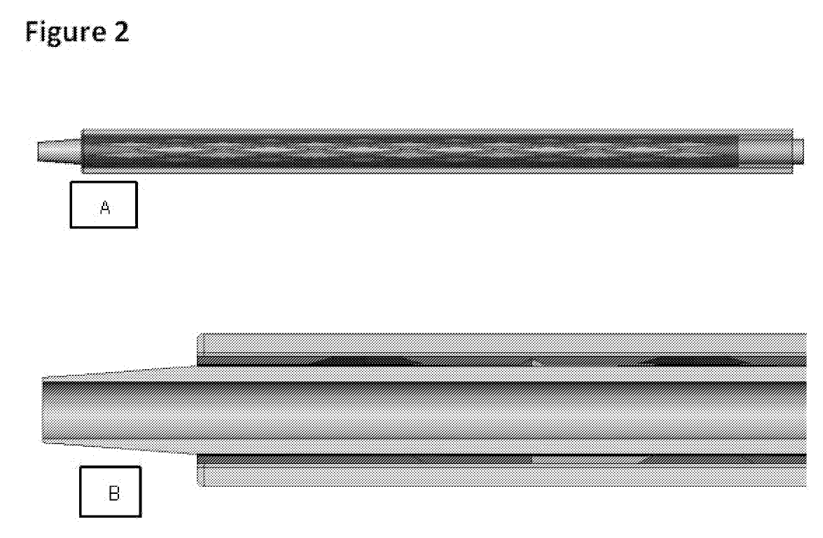

FIG. 2A shows the PreStent Delivery System (PDS) for a self-expanded PreStent.

FIG. 2B shows the system in section view. The catheter has a center lumen for accommodation of a guidewire. The catheter tip is tapered to maximize flexibility so it can easily navigate through tight and tortuous lesions. The lumen of the catheter is about 0.015″-0.016″ (0.381-0.4064 mm) in diameter to accommodate a 0.014″ (0.3556 mm) diameter guidewire. The catheter wall is approximately 0.003″-0.004″ (0.0762-0.1016 mm) thick and is preferably made of polyether block amide (PEBAX), nylon, polyethylene, polyurethane and/or PTFE. A metal braid can be imbedded inside to strengthen the catheter while maintaining its flexibility.

The PreStent is collapsed tight on the catheter OD just proximal to the tapered tip. The overall wall thickness of the collapsed PreStent is about 0.002″-0.004″ (0.0508-0.1016 mm). For the self-expanding PreStent embodiment a retention sheath covers the collapsed PreStent to prevent it from accidentally expanding.

The retention sheath is about 0.003″-0.005″ (0.0762-0.127 mm) thick. It is preferably made of a thin PTFE tube on the inner diameter (ID) and PEBAX on the outer diameter (OD) sandwiching a metal braid in between. The metal braid provides the retention sheath with good hoop strength to contain the PreStent. The overall diameter of the crossing profile is smaller than the typical EPD at 0.035″-0.040″ (0.889-1.016 mm).

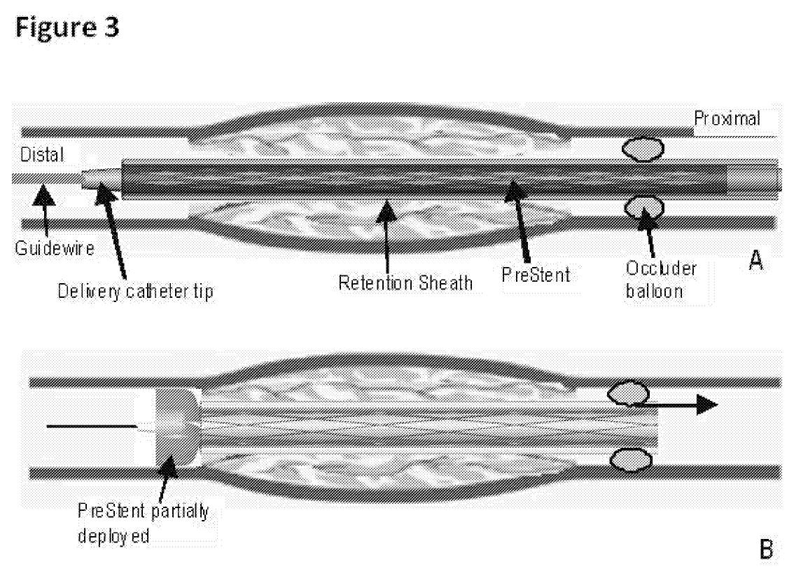

The catheter has a slidable tube with an expandable occlusion element such as a toroidal balloon (see FIG. 3A) on the outside proximal to the PreStent.

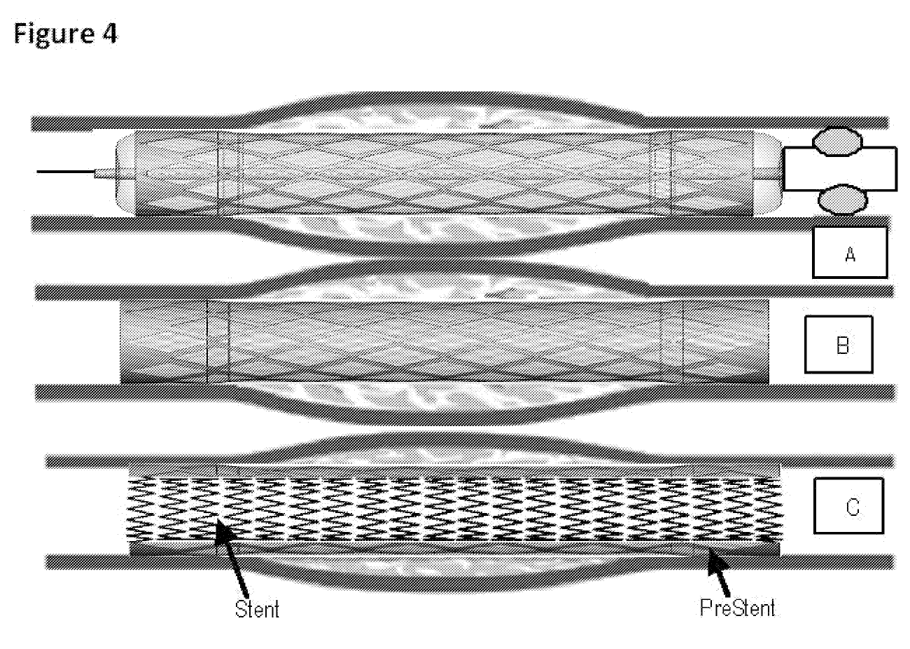

The procedure begins by first deploying the occlusion element and crossing the lesion with the delivery catheter over the guidewire as shown in FIG. 3A. The occlusion element prevents emboli, if any, from being washed distally by blood during crossing. Once the PreStent is several millimeters distal to the lesion, the retention sheath is pulled back to allow the PreStent to expand gradually from its distal end to its proximal end (see FIG. 3B). First, the PreStent distal flare apposes well against the vessel to trap plaque and prevent embolization during the rest of the deployment. The delivery catheter can be taken out after complete deployment of the PreStent. Then, a dilation balloon can be put inside the PreStent and inflated to open up the lumen within the lesion (see FIG. 4A). Finally a stent of choice can be deployed within the PreStent (see FIG. 4C).

This approach allows a physician to operate over their choice guide wire as opposed to the wire incorporated into the typical EPD. This approach also provides a good open lumen for blood flow during catheter exchanges, if necessary, which is better for the patient. Typical EPD either block off blood flow or compromise it.

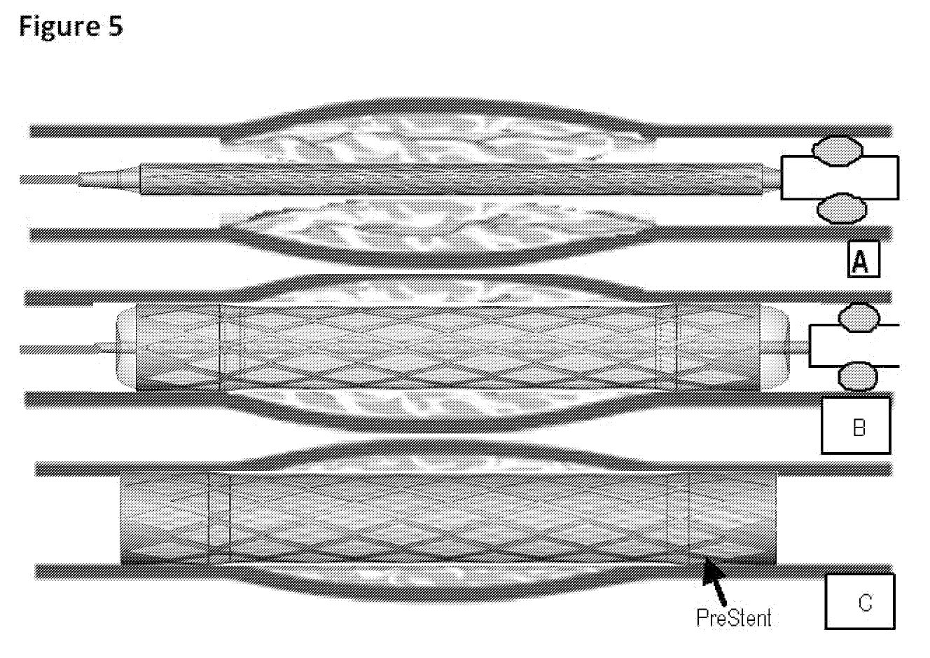

The procedural steps for deploying a balloon-expanded (in contrast to a self-expanded) PreStent are shown in FIG. 5. The PreStent is crimped onto the balloon on the catheter. In this approach, there is no retention sheath required for the delivery system because there is no balloon capable of prematurely or inadvertently expanding on the PreStent itself. Also, in this approach, the predilatation of the lesion can be accomplished by the PreStent deployment balloon which saves procedural time by eliminating the need to exchange in a separate dilatation balloon.

A typical procedure that requires the use of an EPD can benefit from the use of a proximal blood occlusion device while the EPD is crossing the lesion in order to minimize the chance of embolization during the initial crossing. A typical procedure requires a guide catheter or guide sheath that acts as a tunnel for delivering therapeutic devices such as stents inside. These guides or sheaths are usually placed near the proximal region of the target lesion. Bagaoisan (U.S. Published Patent Application No. 20020026145) discloses an occlusion balloon on the tip of the guide catheter. However, the occlusion mechanism and its actuation means on the catheter can add significant bulk. Typical guide catheters and sheaths already need to have their IDs maximized to accommodate bulky therapeutic devices such as stents. The addition of this occlusion mechanism to the outside of the guide also increases the OD and can cause it to be too big, increasing the chance for trauma to a patient in the arterial access port or the vasculature.

One solution to this problem is to put the occluder mechanism on the introducer (Shuttle Select®), selective (Slip-Cath®), or angiography catheter (Headhunter). These catheters usually have a tip that tapers down to a 0.035″ (0.0889 mm) diameter guidewire and are smaller in diameter than the guide catheters or sheaths. They can access a target vessel easier than the guide catheters or sheaths. Physicians already use these catheters to help get the guide catheters or sheath tip near the target lesion. Since this catheter is taken out before introduction of therapeutic devices, it can be bulky on its wall so incorporation of the occlusion mechanism will not change the OD of the device. Its ID can be as small as 0.040″ (1.016 mm) and the OD as big as 0.75″ (19.05 mm). The wall can be 0.017″ (0.4318 mm) thick.

The tapered tip of the introducer, selective, or angiography catheter can be dilatable to accommodate passage of an EPD (i.e. of 0.042″ (1.0668 mm) diameter) through the tip. It can be split and covered with an elastomer (i.e. silicone, isoprene, polyurethane, and/or polyblend) or expandable mesh (i.e. ePTFE) or it can be an expandable coil or mesh covered with an elastomer. The occluding mechanism can be an expandable mesh with an elastomeric cover, an umbrella, a basket, or a balloon. The occlusion mechanism is located near the tip of the catheter.

In using the introducer, selective, or angiography catheter, first the catheter is advanced near the proximal margin of the lesion. Then the occluder is deployed to occlude flow through the lesion. The EPD is then introduced through the ID of the catheter and out the expandable tapered tip to cross the lesion. The EPD is deployed distal to the lesion and the catheter is taken out leaving the guide sheath in place.

Another way to minimize difficulty and embolization with introduction of the EPD is to have a low profile catheter to deploy a cover sheath over the lesion and then predilate with a balloon to open the lesion. Then the EPD can easily and safely cross the opened-up lesion without causing embolization. The cover sheath can then be collected by the delivery catheter and pulled out before deployment of a stent. Similar embolic protection can also be achieved with a toroidal balloon with flares on the ends to cover lesion. The balloon can be inflated to predilate the lesion without embolization, allowing for safe and easy crossing of an EPD. The balloon can be deflated and taken out before stenting.

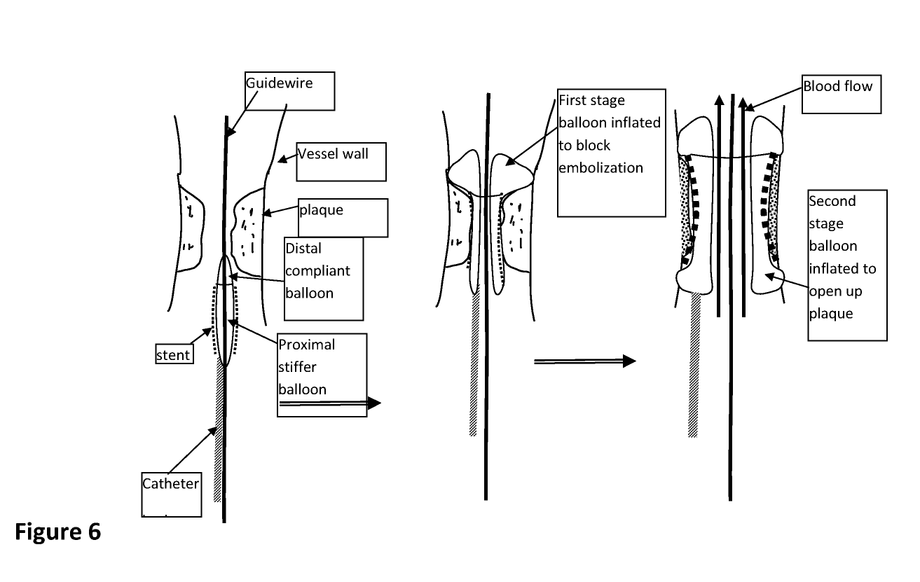

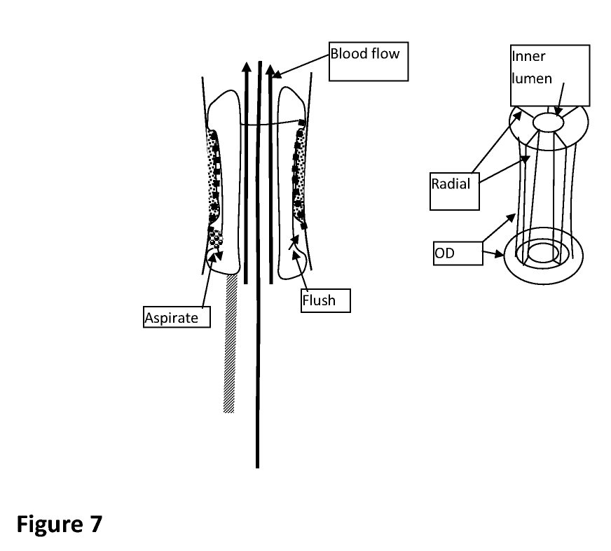

Another approach is to perform direct stenting with a dumbbell-shaped balloon with the stent loaded on the waist of the dumbbell (FIG. 6). The balloon could be multistage (inflatable in multiple distinct stages) where the distal and proximal enlarged ends are first inflated to seal off an embolization path with lower pressure (to not cause injury to the vessel). Subsequently, the central region of the balloon is inflated to deploy the stent and dilate the plaque outward. Preferably, the balloon has a toroidal (donut-shaped) configuration to allow blood flow through the center during the procedure. In an alternative design, the balloon can be replaced by a dumbbell-shaped tube that springs open to seal the lesion ends when the retention sheath is pulled back to deploy both the stent and the tube. The tube can be a stent-like structure (i.e. Nitinol stent strut) covered with an expandable sheath (i.e. of ePTFE, silicone, polyurethane, isoprene or other elastomers). Once the protective sheath and stent are deployed, a balloon catheter can be inserted into the center channel over the guidewire and inflated to dilate the lesion and fully expand the stent. FIG. 7 shows various ways to construct the toroidal balloon and to add aspiration means to the device in order to evacuate emboli trapped by the dumbbell balloon or tube.

What is claimed is:

1. A tube for trapping plaque against a vessel wall, preceding deployment of a stent, comprising:
a proximal end and a distal end;
a radially expandable sheath covering a longitudinal length of the tube; and
a radially expandable frame structure distributed throughout the longitudinal length of the tube configured to hold the sheath open;
wherein said frame structure is composed of a series of discrete helical coils,
said coils circumferentially slidable relative to the sheath, and
the coils on ends of the tube having adjacent legs that overlap.

2. The tube as in claim 1, wherein the sheath is porous.

3. The tube as in claim 2, wherein the sheath has pores that are 10 microns to 80 microns is diameter.

4. The tube as in claim 1, wherein the sheath is made of an elastomer to allow it to expand over a wide range.

5. The tube as in claim 4, wherein the elastomer is selected from the group consisting of: silicone, polyurethane, polyzene-F, and isoprene.

6. The tube as in claim 1, wherein the sheath is viscoelastic.

7. The tube as in claim 1, wherein the sheath does not springback after expansion.

8. The tube as in claims 1, wherein the sheath has a wall thickness of 3 microns to 77 microns.

9. The tube as in claim 1, wherein the sheath comprises thin fibers that are woven, braided, bonded and/or knitted together.

10. The tube as in claim 9, wherein the thin fibers are comprised of material selected from the group consisting of: Nitinol, stainless steel, titanium alloys, tantalum, tungsten alloys, carbon fibers, and glass fibers.

11. The tube as in claim 1, wherein the sheath comprises thin fibers linked together in aligned fashion.

12. The tube as in claim 11, wherein the aligned linked fibers are disposed on an inner diameter of the sheath and are aligned approximately parallel to the longitudinal length of the tube and a longitudinal length of a vessel in which the tube is implanted.

13. The tube as in claim 1, wherein the sheath is made of a biodegradable material.

14. The tube as in claim 13, wherein the biodegradable material of the sheath is selected from the group consisting of: magnesium alloys, hydroxyapatite, polylactic acid (PLA), poly L-lactic acid (PLLA), polyglycolic acid (PGA), polycaprolactone, polyhydroxybutyriate, polydioxanone, polyanhydrides, poly-ortho esters, polyiminocarbonates, polyetheresters, any co-polymers of any of the aforementioned polymers, any blend of any of the aforementioned polymers or co-polymers, silk, modified collagen, and any combination of any of the aforementioned materials.

15. The tube as in claim 1, wherein the sheath is negatively charged.

16. The tube as in claim 1, wherein the sheath is hydrophobic on a surface of an outer diameter and negatively charged on a surface of an inner diameter.

17. The tube as in claim 1, wherein the sheath is coated with or contains an anti-thrombogenic substance.

18. The tube as in claim 1, wherein the sheath is coated with or contains an anti-proliferative substance.

19. The tube as in claim 17, wherein the anti-thrombogenic substance of the sheath is selected from the group consisting of: dextran, heparin, ticlopidine, chlopidogrel, enoxaparin, dalteparin, hirudin, bivalirudin, argatroban, danparoid, TFPI (tissue factor pathway inhibitor), and any combination of the aforementioned substances.

20. The tube as in claim 18, wherein the anti-proliferative substance of the sheath is selected from the group consisting of: Taxol™, Taxan™, Everolimus™, Rapamycin™, rapamycin analogs, antisense dexamethasone, angiopeptin, Batimistat™, Translast™, Halofuginon™, nicotine, acetylsalicylic acid, Tranilast™, and any combination of the aforementioned substances.

21. The tube as in claim 1, wherein the sheath comprises a therapeutic agent selected from the group consisting of: steroids, ibuprofen, antimicrobials, antibiotics (including Actinomycin D), tissue plasma activators, antifibrosis agents, fluoroquinolone, estradiol, and any combination of the aforementioned substances.

22. The tube as in claim 1, wherein the sheath is coated with an endothelialization promoting substance.

23. The tube as in claim 22, wherein the endothelization promoting substance is selected from the group consisting of: vascular endothelial growth factor (VEGF), angiopoietin-1, phosphorylcholine, high density lipoprotein, antibody to receptor CD34, polyzene-F, and any combination of any of the aforementioned substances.

24. The tube as in claim 1, wherein the sheath is comprised of a bioresorbable nonreactive expandable material that is a fluoropolymer.

25. The tube as in claim 1, wherein the sheath comprises a radioopaque substance.

26. The tube as in claim 1, wherein the coils of the frame structure are arranged in pairs and each pair is interconnected by at least one flexible curve.

27. The tube as in claim 1, wherein the coils of the frame structure are self-expanding.

28. The tube as in claim 1, wherein the frame structure is made of shape memory or superelastic materials.

29. The tube as in claim 28, wherein the shape memory or superelastic materials used to form the frame structure are selected from the group consisting of: Nitinol, aliphatic polyesters, polyetherestems L,L-dilactide, diglycolid, and p-dioxanone.

30. The tube as in claim 1, wherein the coils of the frame structure are balloon expandable.

31. The tube of claim 1, wherein the frame structure comprises radioopaque markers on at least some of the coils.

32. The tube as in claim 1, wherein the frame structure is disposed on an inner diameter of the sheath.

33. The tube as in claim 1, wherein the frame is encapsulated within a wall of the sheath.

34. The tube as in claim 1, wherein the sheath comprises an inner layer and an outer layer, and the frame structure is positioned in between said inner layer and said outer layer of the sheath.

35. The tube as in claim 34, wherein the sheath layers are attached between the coils of the frame structure.

36. The tube as in claim 1, wherein the frame structure is made of a biodegradable material.

37. The tube as in claim 13, wherein the frame structure is made of a biodegradable material.

38. The tube as in 37, configured to degrade or be absorbed in 1 month to 6 months.

39. The tube as in claim 36, wherein the biodegradable material is selected from the group consisting of: magnesium alloys, hydroxyapatite, polylactic acid (PLA), polyglycolic acid (PGA), polycaprolactone, polyhydroxybutyriate, polydioxanone, polyanhydrides, poly-ortho esters, polyiminocarbonates, polyetheresters, any blend of the aforementioned polymers, any co-polymers of the aforementioned polymers, modified bone, and any combination of the aforementioned substances.

40. The tube as in claim 1, wherein the material used to form the frame structure is selected from the group consisting of: stainless steel (316L), titanium alloys (Ti6Al4V), tantalum, cobalt chromium, tungsten, and tungsten carbide.

41. A system for trapping plaque against a vessel wall preceding stent deployment comprising:
a plaque-trapping tube having a radially expandable sheath covering the length of a radially expandable frame structure distributed throughout the length of the tube configured to hold the sheath open,
said frame structure composed of a series of discrete, self-expanding helical coils,
said coils circumferentially slidable relative to sheath,
wherein the coils on each end of the tube have overlapping legs adjacent to each other; and
a delivery catheter with a distal end and a proximal end, comprising
a flexible tube having a guidewire lumen inside,
wherein the flexible tube has a region on an outer diameter near a tip at the distal end,
upon which said plaque-trapping tube is collapsed; and
a restraining sheath, configured to restrain said tube in a collapsed state, covering an outer surface of said tube and slidable proximally to allow said tube to expand in a distal to proximal direction as the sheath is retracted.

42. The system of claim 41, wherein a largest diameter of the delivery catheter that crosses the lesion is positioned near the tip at the distal end and is less than 1.1 mm.

43. The system of claim 41, wherein an occlusion balloon is disposed on the delivery catheter proximal to the expandable plaque-trapping tube.

44. A system for trapping plaque against a vessel wall preceding stent deployment comprising:
a plaque-trapping tube having a radially expandable sheath covering the length of a radially expandable frame structure distributed throughout the length of the tube and configured to hold the sheath open,
said frame structure composed of a series of discrete, expandable helical coils,
said coils circumferentially slidable relative to the sheath,
wherein the coils on each end of the tube have overlapping legs adjacent to each other; and
a delivery catheter with a distal end and a proximal end, comprising
a flexible tube having a guidewire lumen inside,
wherein the flexible tube has a region on an outer diameter near a tip at the distal end,
upon which a balloon is attached, and
wherein the plaque-trapping tube is collapseable upon the balloon.

45. The system of claim 44, wherein a largest diameter of the delivery catheter that crosses the lesion is positioned near the tip at the distal end and is less than 1.1 mm.

46. The system of claim 44, wherein an occlusion balloon is disposed on the delivery catheter proximal to the expandable plaque-trapping tube.

47. A method for trapping plaque against a vessel wall preceding stent deployment comprising:
advancing a guidewire across a lesion;
advancing a collapsed, self-expandable tube across the lesion with a delivery catheter such that a distal end of the tube is placed distally to a distal margin of the lesion,
wherein said tube is configured to have a series of discrete self-expanding coils disposed along a length of an expandable sheath;
retracting a restraining sheath to first allow a distal coil of said tube to expand and seal against the vessel wall;
further retracting the restraining sheath to allow all coils of said tube to expand against the vessel wall such that a proximal coil seals against the vessel wall proximal to the proximal lesion margin to completely cover the lesion.

48. The method of claim 47, wherein in the step of further retracting the restraining sheath to allow all coils of said tube to expand against the vessel, the coils expand progressively from a distal end to a proximal end.

49. The method as in claim 47, further comprising the step of expanding an expandable member disposed on the delivery catheter to occlude blood flow, before crossing the lesion with the self-expandable tube.

50. The method as in claim 47, further comprising the step of releasing an anti-proliferative or anti-thrombogenic substance from the expandable sheath.

51. A method for trapping plaque against a vessel wall preceding stent deployment comprising the steps of:
advancing a guidewire across a lesion;
advancing a collapsed, expandable tube across the lesion with a delivery catheter such that a distal end of the tube is placed distally to a distal margin of the lesion,
wherein said tube is configured to have a series of discrete, expandable coils disposed along a length of an expandable sheath;
inflating a dumbbell-shaped balloon to expand a distal coil and a proximal coil of said tube, to expand the tube distally and proximally, to seal against the vessel wall beyond a distal margin and beyond a proximal margin of the lesion, before expansion of the coils within the lesion between the distal margin and the proximal margin.

52. The method as in claim 51, further comprising the step of expanding an expandable member disposed on the delivery catheter to occlude blood flow, before crossing the lesion with the expandable tube.

53. The method as in claim 51, further comprising the step of releasing an anti-thrombogenic or anti-proliferative substance from the expandable sheath.

54. A method for trapping plaque against the vessel wall preceding stent deployment comprising the steps of:
advancing a guidewire across a lesion;
advancing a collapsed, expandable tube across the lesion with a delivery catheter such that a
distal end of the tube is placed distally to a distal margin of the lesion,
wherein said tube is configured to have a series of discrete expandable coils disposed along a length of an expandable sheath;
inflating a first balloon to expand a distal coil and a proximal coil of said tube to expand the tube distally and proximally, to seal against the vessel wall beyond a distal margin and beyond a proximal margin of the lesion, before expansion of the coils within the lesion between the distal margin and the proximal margin; and
inflating a second balloon to expand the coils within the lesion.

55. A tube for trapping plaque against the vessel wall preceding stent deployment comprising:
a self-expandable mesh structure distributed throughout the length of the tube;
one or more coil positioned at least on each end of the tube;
wherein said mesh is attached to the coils on its ends where adjacent legs of the coils overlap.

56. The tube as in claim 55, wherein the mesh and the coils are formed of the same fibrous material such that a frame element comprising the coils and a sheath element comprising the mesh are combined.

57. The tube as in claim 56, wherein the fiber thickness is 0.0005″ to 0.002″ (0.0127-0.0508 mm).

58. The tube as in claim 55, wherein the mesh has a pore size of 10-50 microns.

59. The tube as in claim 55, wherein the mesh has pore size of 5-80 microns.

60. The tube as in claim 55, wherein the mesh is constructed of wires braided together.

61. The tube as in claim 55, wherein at least one of the mesh component and the coil component is biodegradable, bioabsorbable, and/or bioerodable.

62. The tube as in claim 61, configured to degrade, be absorbed, and/or erode in 1 month to 6 months.

63. A method for inserting an embolic protection device to trap plaque against a vessel wall preceding deployment of a stent comprising the steps of:
(i) inserting a guide sheath across a lesion;
(ii) advancing an introducer, selective, or angiography catheter having an expandable tapered tip near a proximal margin of a lesion;
(iii) deploying an occluder element to occlude flow through the lesion;
(iv) introducing an embolic protection device through an inner diameter of the catheter and out the expandable tapered tip of the catheter to cross the lesion;
(v) deploying the embolic protection device distally to the lesion; and
(vi) removing the catheter while leaving the guide sheath in place.

64. The method of claim 63, wherein in the step of introducing an embolic protection device out the expandable tapered tip of the catheter, the tip of the catheter is split to accommodate passage of the embolic protection device.

65. The method of claim 63, wherein the tip of the catheter is composed of expandable coils or expandable mesh, wherein in the step of introducing an embolic protection device out the expandable tapered tip of the catheter, the tip of the catheter is dilated to stretch the coils or mesh and accommodate passage of the embolic protection device.

66. A method for inserting an embolic protection device to trap plaque against a vessel wall preceding deployment of a stent comprising the steps of:
(i) inserting a low profile catheter to deploy a cover sheath over the lesion;
(ii) predilating the lesion with a balloon to open the lesion;
(iii) inserting a delivery catheter to deliver the embolic protection device across the lesion; and
(iv) collecting the cover sheath with the delivery catheter and removing the cover sheath.

67. The method of claim 66, wherein the balloon has a toroidal configuration and flares outward on each end.
